(19) United States
(12) Patent Application Publication
(10) Pub. No.: US 2011/0097745 A1
(43) Pub. Date: Apr. 28, 2011

(54) Method For Detecting Microbes

(75) Inventor: Nirit Ulitzur, Kiryat-Tivon (IL)

(73) Assignee: CHECK LIGHT LTD., Kiryat-Tivon, IL (US)

(21) Appl. No.: 12/673,026

(22) PCT Filed: Aug. 11, 2008
(86) PCT No.: PCT/IL08/01103

§ 371 Date: Feb. 15, 2010

Related U.S. Application Data
(60) Provisional application No. 60/935,435, filed on Aug. 13, 2007.

(51) Int. Cl. G01N 33/569; G01N 33/50; C12M 1/34

(52) U.S. Cl. 435/7.37; 435/7.32; 435/288.7

(57) ABSTRACT

The invention provides means and methods for determining the presence of infecting microbes in a liquid. The method comprises steps of introducing the liquid to the sample container of a microbial analyzer, the sample container further containing nutrients. Other steps include carrying out a first incubation of said specimen, adding bioluminescent tester microbes to the specimen, carrying out a second incubation of the specimen and measuring bioluminescence, thereby determining said presence and/or concentration of infecting microbes.

### FIELD OF THE INVENTION

The invention relates to methods for detecting microbes such as bacteria or fungi.

### BACKGROUND OF THE INVENTION

Most methods for the detection of bacteria in a specimen require preparing a primary culture of the specimen on agar plates, preparing an inoculum from a single colony and assessing the number of bacteria in the inoculum at the end of an incubation period. When the number of bacteria is assessed qualitatively by visual examination 24-48 hours of incubation are usually required. Quantitative methods for assessing the number of bacteria, for example, photometric measurement of turbidity, radidmetric detection of bacterial metabolites, or fluorometric detection of hydrolyzed fluorogenic substrates require an incubation time of a few hours (see e.g C. Thornsbeny et al., J Clin. Microbiol. 12:375 (1980); F. S. Nolte et al., J. Clin. Microbiol., 26:1079 (1988); J. L. Staneck et, al., J Clin. Microbiol., 22:187(1985); P. G. Beckwith et al., J Clin. Microbiol., 15:35 (1982).

U.S. Pat. No. 5,885,791 discloses determining the amount of a nutrient such as glucose present in a culture at the end of an incubation in order to determine the concentration of microbes in the culture. The nutrient is detected at the end of the incubation using an enzymatic assay producing a detectable color.

The intensity of the color is positively-correlated with the concentration of the nutrient at the end of the incubation, which in turn is negatively correlated with the concentration of the microbes in the culture.

### SUMMARY OF THE INVENTION

While the present invention is described with reference to the detection of bacteria, it should be understood that the invention may be applied to the detection of other types of microbes, such as fungi.

The present invention provides a method for quantitatively determining the presence of microbes such as bacteria in an inoculum obtained either from a colony on an agar plate or directly from a liquid sample such as water of urine. In the case of a liquid sample, the concentration of the bacteria in the sample may optionally be raised by centrifuging the sample so as to sediment bacteria and resuspending the bacteria in a smaller volume of liquid, or by filtering the sample so as to collect bacteria on the filter and resuspending the collected bacteria in a smaller volume of liquid.

In accordance with the invention, bacterial nutrients are added to the inoculum. Any bacteria in the inoculum, referred to herein as “infecting bacteria” are allowed to consume the nutrients during a first incubation. At the end of the first incubation, a population of detectable bacteria, referred to as “tester bacteria” are added to the inoculum. The tester bacteria can be of any strain capable of producing a sensible signal such as a strain of luminous bacteria. The tester bacteria may be wild type, auxotrophic mutants or recombinant and are preferably added to the inoculum in a starved state.

The tester bacteria are incubated in the inoculum during a second incubation at the end of which, the number of infecting bacteria present in the inoculum is assessed by measuring the intensity of the signal produced by the tester bacteria. In the case of luminous tester bacteria, for example, the intensity of the light emitted by the bacteria is detected using a luminometer or by exposure of the inoculum to visible light film. The intensity of the measured signal is positively correlated with the concentration of nutrients in the inoculum at the beginning of the second incubation which, in turn, is negatively correlated with the concentration of the infecting bacteria in the inoculum at the beginning of the first incubation. Therefore, a relatively intense signal produced by the tester bacteria is associated with an inoculum containing a low concentration of infecting bacteria, and vice versa. The term “nutrients” hereinafter refers to: amino acids and small peptides, vitamins, nucleotides, fatty acids, glucose and sugars. Some embodiments of the present invention provide means and methods for detection of amino acids and peptides by sensor (tester) bacteria carrying the Lux-I deleted lux system of Vibrio fischeri that were pre-starved for carbon source or are defective in their ability to synthesize a certain amino acid.

Vitamins.

Some embodiments of the present invention provide means and methods for detection of a vitamin by sensor (tester) bacteria carrying the Lux-I deleted lux system of Vibrio fischeri are mutated in their ability to generate that specific vitamin or luminescent bacteria mutants selected for their inability to generate that specific vitamin. It is acknowledged herein that E. coli strain 215-B12 requiring mutant), E. coli lysine-requiring mutant (ATCC 23812), methionine-requiring mutant (ATCC 23798); E. coli thiamine & adenine-requiring mutant (ATCC 23804) carrying the Lux-I deleted lux system of Vibrio fischeri and others are provided as tester strains.

Nucleotides.

Further embodiments of the present invention provide means and methods for detection of nucleotides by sensor (tester) bacteria carrying the Lux-I deleted lux system of Vibrio fischeri that are mutated in their ability to generate that specific nucleotide or luminescent bacteria mutant selected for their inability to generate that specific nucleotide.

Glucose

Further embodiments of the present invention provide means and methods for detection of glucose by sensor (tester) bacteria carrying the Lux-I deleted lux system of Vibrio fischeri or native luminescent bacteria that were pre-starved for carbon source.

Fatty Acids

Further embodiments of the present invention provide means and methods for detection of specific fatty acids by sensor (tester) bacteria carrying the Lux-I deleted lux system of Vibrio fischeri that are mutated in their ability to generate that specific fatty acid or luminescent bacteria mutant selected for their inability to generate that specific fatty acid.

Several luminous bacterial strains and recombinant bacteria carrying the lux system are known. A luminous bacterium is a self maintaining luminescence unit that, under proper conditions, emits a level of luminescence that may reach 5×10⁴ quanta/sec/cell.

The luminescence of a single cell is thus readily determined by a photon-counter, while the luminescence of a bacterial suspension of only a few hundred cells per milliliter can be determined with a simple luminometer.

The invention may be used to determine the susceptibility of infecting bacteria to a particular treatment (such as antibacterial agents) by subjecting the inoculum to the treatment during the first incubation. The number of metabolically active bacteria at the end of the incubation is determined in accordance with the invention and compared to a control in which the treatment is omitted. If the signal produced by the tester bacteria is substantially higher in the treated inoculum than in the untreated control, for example, three-fold higher, then the infecting bacteria may be concluded to be susceptible to the treatment.

For example, the invention may be used to determine the susceptibility of the infecting bacteria to an antibiotic drug by adding the drug to the inoculum during the first incubation. In order to avoid any detrimental effect of the antibiotic drug on the tester bacteria, a tester bacteria resistant to that antibiotic is preferably used. For example, the E. coli GlnA auxotrophic mutant (ET 12558), carrying the lux I-deleted lux system of Vibrio fischeri is a luminous bacterial strain that shows resistance to Ampicillin, Tetracycline, and Kanamycin due to the presence of the natural plasmid RP4, and additional resistance to Chloramphenicol due to the presence of the pACYC184 plasmid (that carries the lux system). Since the development of luminescence requires only 30-45 minutes under non-growing conditions, and most antibiotics, especially betalactams, show no significant activity during this time period, a luminous tester bacteria not resistant to the antibiotic may also be used.

The method may also be used to distinguish between Gram-positive and Gram-negative bacterial strains. In this case, the cationic detergent CTAB (Cetyltrimethylammonium bromide) is added to the inoculum during the first incubation. Gram-positive bacteria are more sensitive than Gram-negative bacteria to low concentrations of CTAB.

Thus, in one aspect, the invention discloses a method for the determination of the presence and optionally concentration of an infecting microbe in a liquid specimen, the method comprising steps of
a. obtaining a microbial analyzer
b. introducing the liquid to the sample container of the microbial analyzer. The aforementioned sample container further contains nutrients
c. carrying out a first incubation of the specimen
d. adding the bioluminescent tester microbes to the specimen
e. carrying out a second incubation of the specimen
f. measuring bioluminescence thereby determining the presence and/or concentration of the infecting microbes

In its second aspect, the above defined method is disclosed wherein the infecting microbe is a bacterium.

In another aspect, the above defined method is disclosed wherein the liquid to be tested is selected from a group consisting of urine, water, saline, plasma, blood or blood products.

In another aspect, the above defined method is disclosed wherein the liquid is selected from a group consisting of mains supply water, drinking water, tap water, bottled water, food process water, medicinal process water, reclaimed, agricultural irrigation water.

In another aspect, the above defined method is disclosed wherein the liquid is a beverage.

In another aspect, the above defined method is disclosed wherein the above defined method comprises steps of pre-concentrating the microbes.

In another aspect, the above defined method is disclosed wherein the specimen is prepared from water.

In another aspect, the above defined method is disclosed wherein the concentration comprises steps selected from the group consisting of: centrifuging the microbes, resuspending them in a liquid, filtering the tested liquid, collecting the microbes on the filter and resuspending the filtered microbes in a liquid.

In another aspect, the above defined method is disclosed wherein the tester bacteria are of the GlnA mutant strain of E. coli (ET 12558) carrying the Lux-I deleted lux system of Vibrio fischeri.

In another aspect, a method is disclosed for determining the susceptibility of microbes in a liquid specimen to a substance, the method comprising steps of:

adding the substance to the specimen, determining the concentration of microbes in the specimen by the above defined method and comparing the concentration of the microbes in the specimen to the concentration in a control specimen to which the substance was not added. A lower concentration of microbes in the specimen than that in the control specimen indicating susceptibility of the microbes to the substance. In another aspect of the invention, a method for determining a minimum inhibitory concentration of a substance and of microbes in a liquid specimen is disclosed as follows:

The susceptibility of the microbes to the substance for each of a plurality of concentrations is determined according to the method defined above and a minimal concentration of the substance to which the bacteria are susceptible is determined.

In another aspect of the invention, a method for determining the susceptibility of microbes in a liquid specimen to a substance, is disclosed wherein the substance used is cetyltrimethyl-ammonium bromide.

In another aspect of the invention a method for determining whether microbes in a liquid specimen are Gram positive or Gram-negative is disclosed. The method comprises determining the susceptibility of the microbes as defined above. The aforementioned method further comprises steps of adding a substance that selectively inhibits Gram positive or Gram negative bacteria to the first or second incubation.

In another aspect of the invention, a system is provided for determining the presence of an infecting microbe in a liquid specimen. The system comprises a microbial analyzer, the analyzer further comprising
i. a reaction chamber containing nutrients for carrying out a first incubation and a second incubation of the liquid specimen.
ii. reagent containers for containing and introducing buffers and reagents into the reaction chamber
iii. a light detector adapted for detecting bioluminescence during the incubations

In some embodiments of the invention nutrients are added to the reaction chamber from a reagent chamber with the buffer.

The system further comprises a reagent, wherein the reagent comprises tester bacteria of an E. coli strain (ET 12558) carrying the Lux-I deleted lux system of Vibrio fischeri. The aforementioned strain is characterized by luminescing in proportion to the concentration of the nutrients remaining at the beginning of the second incubation.

In another aspect of the invention, the aforementioned system is provided wherein the reagent comprises tester bacteria of a luminescent mutant (such as aldehyde-requiring or myristic acid-requiring mutants of Vibrio harveyi). These mutants are further characterized by only luminescing in the presence of a breakdown product of a substrate of a surface enzyme of the infecting microbes. The luminescing of the mutants is in proportion to the concentration of the breakdown product at the beginning of the second incubation.

The substrate of the infecting microbes is provided to be added to the first incubation.

In another aspect of the invention, a system is provided for determining the presence of an infecting microbe in a liquid specimen as described above. The system is additionally provided with means of automatic or semi automatic sample preparation.

In another aspect of the invention, a system is provided for determining the presence of an infecting microbe in a liquid specimen as described above. The system is further provided with means of data processing and transmission.

In another aspect of the invention a kit for determining the presence of an infecting microbe in a liquid specimen is provided. The kit comprises
a. a microbial analyzer, the aforementioned analyzer further comprising
i. a reaction chamber for carrying out a first incubation and a second incubation of the liquid specimen
ii. reagent containers for containing and introducing buffers and reagents into the reaction chamber
iii. a light detector adapted for detecting luminescence during the aforementioned incubations
b. a reagent, wherein the reagent comprises tester bacteria of an E. coli strain (ET 12558) carrying the Lux-I deleted lux system of Vibrio fischeri, the strain characterized by luminescing in proportion to the concentration of nutrients remaining at the beginning of the second incubation.

In another aspect of the invention, the aforementioned kit is provided as defined above, wherein the reagent further comprises tester bacteria of a luminescent mutant. The aforementioned mutant is further characterized by only luminescing in the presence of a breakdown product of a substrate of a surface enzyme of the infecting microbes, the aforementioned luminescing in proportion to the concentration of the breakdown product at the beginning of the second incubation. The kit also includes the substrate of the aforementioned infecting microbes.

In another aspect of the invention the aforementioned kit is provided wherein the nutrients are selected from a group consisting of amino acids, peptides, fatty acids, vitamins, nucleotides and sugars. In this aspect of the invention, the sensor bacteria such as naturally selected luminescent mutants or E. coli strain (ET 12558) carrying the Lux-I deleted lux system of Vibrio fischeri are additionally characterised as mutants lacking the ability to generate said selected nutrient. It is acknowledged herein that E. coli strain 215-B12 requiring mutant), E. coli lysine-requiring mutant (ATCC 23812), methionine-requiring mutant (ATCC 23798); E. coli thiamine & adenine-requiring mutant (ATCC 23804) carrying the Lux-I deleted lux system of Vibrio fischeri are provided as tester strains. It is further acknowledged herein that numerous other auxotrophic and recombinant mutants carrying the Lux-I deleted lux system of Vibrio fischeri are well within the scope of the present invention, especially when used for the detection of microbes as described herein.

Moreover, in another aspect of the invention the aforementioned kit is provided further comprising;
a. a reagent comprising tester bacteria of a luminescent dark mutant, said mutant further characterized by only luminescing in the presence of a breakdown product of a substrate of a surface enzyme of said infecting microbes, said luminescing in proportion to concentration of said breakdown product at the beginning of said second incubation
b. said substrate of said infecting microbes

In yet another aspect of the invention the aforementioned kit is provided wherein the breakdown product is defined as a substrate of an intracellular enzyme released from the infecting cells by osmotic shock.

In another aspect of the invention, a kit is provided as defined above wherein the mutant is a myristic acid-requiring mutant of Vibrio harveyi or an aldehyde-requiring mutant of Vibrio harveyi.

### BRIEF DESCRIPTION OF THE DRAWINGS

The accompanying drawings, which are incorporated in and form a part of this specification, illustrate embodiments of the invention and, together with the description, serve to explain the principles of the invention (BLT-Bioluminescence Test):

FIG. 1 shows the determination of antibiotic susceptibility of various bacterial strains incubated in a defined media in accordance with the invention;

FIG. 2 shows the correlation between incubation time and concentration of infecting bacteria.

FIG. 3 depicts the linear response between contaminating cells concentration and generated luminescence in an embodiment of the invention.

FIG. 4 describes a test in which a water sample was preincubated with polymyxin B (PMB-1.25 mg/L) and PCDM (25 mg/L) with and without 10⁵ cells/ml isolated from sewage in a preferred embodiment of the invention.

FIG. 5 shows a preferred embodiment of the present invention, providing automatic means and methods for detecting microbes in a fluid as herein described.

FIG. 6 shows a block diagram of a preferred embodiment of the present invention, providing automatic means and methods for detecting microbes in a fluid as herein described

### EXAMPLES

### Example 1

### Determining the Antibiotic Susceptibility of Bacteria

The invention was used to determine the susceptibility of several strains of 30 infecting bacteria to various antibiotics. A single colony of the infecting bacteria from a pre-cultured plate or a liquid broth of mid-logarithmic phase bacteria was suspended or added to Medium 1 (0.1% glucose, 50 ppm yeast extract (Difco) in Davis Buffer) to yield a final cell concentration of 10⁶ cells/mL, 0.1 mL aliquots of this suspension were dispensed in test tubes with or without the tested antibiotic agent. The tubes were then incubated 90-120 minutes at 37 degrees Celsius. 0.1 mL of 10⁶ cells/mL E. coli Gln in Medium 2 (16% ASW, 80 mM MgSO₄ 16 μ/mL folic acid, 16% sucrose, 32 mM KH₂PO₄, _{P}H 5.5, 10-50 ng/mL autoinducer) were added and the tubes were incubated at 28 degrees Celsius for 30-60 minutes.

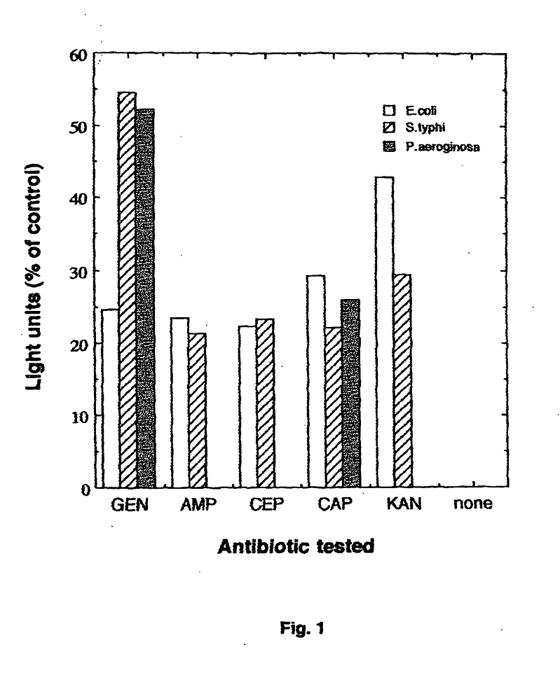

The pH of 5.5 and the presence of sucrose and MgSO₄ in Medium 2 drastically reduce the activity of aminoglycosides and quinolones. Folic Acid antagonises the activity of Sulpha drugs. The inoculum was diluted to 10-20 fold in the assay medium thus reducing any inhibitory effect of the tested antibiotic on the tester bacteria. At the end of the second incubation, the luminescence level in each sample was determined using a luminometer. The results are shown in FIG. 1.

For comparison, Table 1 shows the susceptibility of the infecting bacterial strains to the antibiotics as determined by the overnight disk diffusion test. Infecting bacteria (10⁶ cells/ml from overnight grown cultures) were spread on LA plates on which 15 mm disks, saturated with various antibiotics, had been placed. After incubating the plates at 37 degrees Celsius for 16 hrs the zone of inhibition was determined for each strain. (Abbreviations used in FIG. 1a and Table 1a: CAP—Chloramphenicol; KAN—Kanamycin; AMP—Ampicillin; CEP—Cephamezin GEN—Gentamycin).

As can be seen, antibiotic susceptibility determined by the method of the invention is in agreement with the results obtained in the disk diffusion test.

TABLE 1

| | Antibiotics conc. | | | | |
|---|---|---|---|---|---|
| | CAP | KAN | AMP | CEP | GEN |
| Strain | (30 ppm) | (30 ppm) | (40 ppm) | (30 ppm) | (10 ppm) |
| E. coli | 25 mm | 25 mm | 40 mm | 30 mm | 15 mm |
| (W4110) | | | | | |
| S. | 30 mm | 20 mm | 30 mm | 40 mm | 20 mm |
| typhimirium | | | | | |
| (772) | | | | | |
| P. | 15 mm | — | — | — | 20 mm |
| aeroginosa | | | | | |

### Example 2

### Determination of Minimum Inhibitory Concentration (MIC)

The system of Example 1 was used for the determination of the MIC of Gram-positive (S aureus) and Gram-negative (S. typhi) infecting bacteria to various drugs. Each drug was serially diluted in 0.1 mL Medium 1 (0.1% glucose, 25 ppm yeast in Davis buffer) and incubated with the infecting bacteria (prepared as described in Example 1) for 90 minutes at 37 degrees Celsius. 0.15 ml of Medium 2 containing the tester bacteria (final concentration of 10⁶ cells/mL per tube) were then added to each tube. Following a 45 min. incubation at 28 degrees Celsius, the luminescence was determined.

Luminescence levels were normalized to the level in the absence of the antibiotic. MIC is calculated as the lowest drug concentration which inhibits growth to such an extent as to generate a two-fold increase of luminescence by the tester bacteria above the control. As shown in Table 2, the MIC for Gentamycin (˜3.12-6.2 ppm; S. aureus), Kanamicin (˜1 ppm; S. typhi), and Chloramphenicol (˜3 ppm and ˜1.5 ppm; S. typhi, S. aureus, respectively) were evaluated for each strain.

Table 2 also presents the MIC values obtained using a standard assay in which infecting bacteria (10⁵ cells/mL) were incubated in LB with serial double dilutions of the drugs in liquid broth and left at 37 degrees Celsius overnight. The tubes were then inspected for visible growth (G), slight growth (SG), or no growth (NG).

The results obtained by the method of the invention were in agreement with the standard test.

TABLE 2
MIC determination for S. typhi and S. aureus using
the standard overnight liquid broth test and BLT
Table 2: MIC determination for S. typhi and S. aureus using
the standard overnight liquid broth test and BLT
S. typhi

| Kanamicin | | | Chloramphenicol | | |
|---|---|---|---|---|---|
| (ppm) | LU* | Growth | (ppm) | LU* | Growth |
| 30 | 6.27 | NG | 50 | 5.84 | NG |
| 15 | 5.79 | NG | 25 | 6.84 | NG |
| 7.5 | 3.13 | NG | 12.5 | 5.42 | NG |
| 3.75 | 2.45 | NG | 6.25 | 3.61 | NG |
| 1.875 | 2.3 | SG | 3.1 | 1.9 | SG |
| 0.937 | 1.82 | SG | 1.5 | 0.74 | G |
| 0.468 | 1.49 | G | 0.78 | 0.63 | G |
| 0.234 | 1.62 | G | 0.39 | 0.72 | G |
| 0.117 | 1.4 | G | 0.19 | 0.7 | G |

S. aureus

| Gentamicin | | | Chloramphenicol | | |
|---|---|---|---|---|---|
| (ppm) | LU* | Growth | (ppm) | LU* | Growth |
| 50 | 6.5 | NG | 100 | 70.1 | NG |
| 25 | 3.4 | NG | 50 | 17.6 | NG |
| 12.5 | 3.6 | NG | 25 | 11.5 | NG |
| 6.25 | 3.1 | NG | 12.5 | 7.2 | NG |
| 3.125 | 1.5 | G | 6.25 | 5.2 | NG |
| 1.562 | 1.3 | G | 3.125 | 2.8 | NG |
| 0.781 | 1.2 | G | 1.562 | 1.3 | G |
| 0.39 | 1.2 | G | 0.781 | 0.8 | G |

*Fold increase over control (no antibiotics added).

### Example 3

### Differentiation between Gram-Positive and Gram-Negative Bacteria

Gram-positive bacteria are more sensitive than Gram-negative bacteria to low concentrations of the cationic detergent CTAB. Overnight grown cultures of different strains of infecting bacteria were washed and suspended in saline. About 10⁵ cells/well were aliquoted in 0.1 mL Medium 1 (Davis, 0.1% glucose, 25 ppm YE) in microtiter plates with or without 3 ppm of CTAB. After 2 hrs at 37 degrees Centigrade/Celsius?, 0.15 mL of Medium 2 (+10 ng/mL autoinducer) were added to each well, together with 10⁶ cells/mL lux-cells. Luminescence was recorded after 60 min.

As can be seen in Table 3, the presence of CTAB hindered the ability of the Gram positive strains (S. aureus., and Streptococcus sp.) to consume the nutrients provided, leading to higher level of luminescence by the tester bacteria. The ability of the Gram-negative strains to consume nutrients was essentially unaffected by the presence of CTAB.

TABLE 3
Differentiation between Gram positive
and Gram negative strains using CTAB

| | | |
|---|---|---|
| Strain | +CTAB (3 ppm) | No CTAB |

| | | | | |
|---|---|---|---|---|
| E. coli (W4100) | 52 | LU* | 19 | LU |
| S. typhi (772) | 64 | LU | 14 | LU |
| S. aureus | 80,405 | LU | 324 | LU |
| Streptococcus sp. | 10,154 | LU | 2,133 | LU |
| P. aeroginosa | 111 | LU | 98 | LU |
| Control (no infecting bacteria) | 91,040 | LU | 106,263 | LU |

*LU = Light Units

### Example 4

### Detection of Bacteria in Urine and Their Antibiotic Susceptibility

Quantitative bacterial counts greater than 10⁵ colony forming units/mL are often used as clinical markers for significant bacteriuria while a lower level of colony forming units/mL is usually considered to be due to artifactual contaminants. However, it has been reported that “low count” bacteriuria defined as 10² to 10⁴ colony forming units/mL are statistically more frequent in women with urine complaints than in asymptomatic women. In addition, <10² colony forming units/mL in catheterized patients is considered significant bacteriuria. Sixteen fresh urine samples obtained from a local clinic were tested for the presence of bacteria in three methods: colony formation on a Luria agar plate, the catalase activity test (Uriscreen commercial kit—Savion Ltd.), and the method of the present invention. The number of bacteria in the urine samples was assessed without preincubation in Medium 1, by adding tester lux-cells in Medium 2 to 1% urine (diluted in Medium 2) and measuring luminescence after a 30-60 min. incubation at room temperature (20-28° C.). The results are shown in Table 4. Using colony count as standard, samples 2 and 16 were sterile; samples 1, 3, 4, 7, 8, 12 and 15 contained more than 10⁵cells/mL; and samples 9, 13 and 14 had a mixed population of cells suggestive of a post urination contamination. The catalase test detected three out of the seven while the method of the present invention detected four out of seven (lowest light levels—marked in bold) of the heavily contaminated samples. It should be noted that the catalase activity test cannot differentiate between post-urination contamination and true bacteriuria.

The method of the present invention, on the other hand, identified these cases since, in this case, no consumption of urine nutrients occurs unless the samples were at >20 degrees Celsius for a few hours. In addition, strains lacking catalase activity, such as Enterococci, cannot be identified by the catalase activity test.

TABLE 4
Determination of bacterial load in urine samples using the Commercial
Savion-catalase kit, BLT, and overnight colony count.

| | Colony count | Catalase activity | BLT |
|---|---|---|---|
| Urine sample # | (cells/ml) | (foam) | (light units) |
| 1 | 10⁶ | ++ | 196 |
| 2 | <10² | − | 14,000 |
| 3 | 10⁶ | − | 1,110 |
| 4 | 10⁶ | − | 2,211 |
| 5 | 1.5 × 10⁴ | − | 37,000 |
| 6 | 2.5 × 10⁴ | − | 91,000 |
| 7 | 8 × 10⁵ | ++ | 248,000 |
| 8 | 10⁶ | +++ | 8,000 |
| 9 | 10⁵ mix | ++ | 1,316,000 |
| 10 | 8 × 10⁴ | +− | 59,000 |
| 11 | 3 × 10³ | − | 565,000 |
| 12 | 10⁵ | − | 95,000 |
| 13 | 1.2 × 10⁵ mix | +− | 375,000 |
| 14 | 1.2 × 10⁵ mix | − | 106,000 |
| 15 | 8 × 10⁵ | − | 17,000 |
| 16 | <10² | ++ | 68,000 |
| Suspected | | 3/7 | 4/7 |
| Bacteriuria | | | |
| (>10⁵ cells/ml) | | | |
| Mixed culture | | 2/3 | 0/3 |
| Sterile sample | | 1/2 | 0/2 |

### Example 5

### Determination of Bacterial Antibiotic Susceptibility

Six fresh urine samples were obtained from a local clinic and examined for bacteriuria (>10⁵ cells/mL urine) and the antibiotic susceptibility of the infecting bacteriuria. A tube containing a an antibiotic mixture that would eliminate all infecting bacteria but not effect the lux cells (Ampicillin, Kanamycin, Tetracyclin, and Chloramphenicol) served as a control.

Each urine sample was diluted in Medium 1 to yield a final concentration of 3%. 0.1 mL of the diluted samples were then incubated for 3 hrs. at 37 degrees Celsius in a microtitre plate, with or without an antimicrobial substance at the concentrations given in Table 5).

An equal volume of Medium 2 containing 10 ng/ml autoinducer and supplemented with 10⁶ of the lux cells was then added to each well. The microtiter plate was then incubated at 28 degrees/Celsius and the luminescence was measured using a plate reader luminometer.

Luminescence was measured in the absence of antibiotics, in the presence of a mixture of antibiotics and in the presence of a single antibiotic from the mixture.

The results are shown in Table 5c. A standard analysis (urine culture colony count) was performed to evaluate bacterial load (Table 5a) and antibiotic susceptibility (Table 5b).

Luminescence was measured in the presence of a mixture of antibiotics, in the presence of a mixture of antibiotics, and in the presence of a single antibiotic from the mixture. The results are shown in Table 5c. A standard analysis (urine culture colony count) was performed to evaluate bacterial load (Table 5a) and antibiotic susceptibility (Table 5b).

When the ratio of the luminescence measured in the presence of the antibiotic mix to that in the absence of antibiotics does not exceed a first predetermined constant (eg. 2), then the concentration of the infecting bacteria is less than 10⁵ cells/mL. When the ratio of the luminescence measured in the presence of the single antibiotic to that measured in the presence of the antibiotic mix exceeds a second predetermined constant (e.g. 3) then the infecting bacteria are susceptible to the antibiotic.

The results are shown in Table 5 (d).

TABLE 5
5a: Colony plate count

| Sample # | 1 | 2 | 3 | 4 | 5 | 6 |
|---|---|---|---|---|---|---|
| Cells/ml × | 250 | 1000 | 1000 | 350 | 200 | 1000 |
| 1000 | | | | | | |

5b: Antibiotics disc inhibition zone (mm)

Urine sample #

| Antibiotics | 1 | 2 | 3 | 4 | 6 | 6 |
|---|---|---|---|---|---|---|
| Gentamicin (10 ppm) | 4 | 5 | 6 | 2 | 5 | 5 |
| Ampicillin (40 ppm) | 0 | 0 | 5 | 7 | 0 | 5 |
| Penicillin G (40 ppm) | 0 | 0 | 3 | 7 | 0 | 2 |
| Cefuroxime (30 ppm) | 6 | 7 | 5 | 5 | 7 | 4 |
| Orbenil (10 ppm) | 0 | 0 | 0 | 6 | 0 | 0 |
| Cephamezin (30 ppm) | 7 | 5 | 7 | 5 | 5 | 6 |
| Chloramphenicol | 5 | 4 | 7 | 5 | 6 | 6 |
| (30 ppm) | | | | | | |
| Kanamicin (30) | 5 | 5 | 5 | 2 | 5 | 1 |

5c: BLT

| | # urine | | | | | |
|---|---|---|---|---|---|---|
| Antibiotics | 1 | 2 | 3 | 4 | 5 | 6 |
| Gentamicin | 53 | 44 | 30 | 36 | 11.5 | 35 |
| Ampicillin | 1 | 0.144 | 5.11 | 30 | 0.42 | 18 |
| Penicillin G | 0.5 | 0.93 | 4 | 30 | 0.417 | 11 |
| Cefuroxime | 55 | 1.36 | 2.4 | 31 | 8.6 | 0.76 |
| Orbenil | 0.8 | 0.087 | 0.76 | 30 | 0.63 | 0.18 |
| Cefamezine | 61 | 2.3 | 21.8 | 30 | 16 | 36.8 |
| Chloram- | 62 | 46 | 36 | 31.2 | 13 | 41 |
| phenicol | | | | | | |
| Kanamicin | 64 | 42 | 49 | 30 | 16 | 38 |
| Antibiotics | 62 | 47 | 85 | 32 | 16 | 41 |
| mix control | | | | | | |
| No antibiotics | 0.317 | 0.062 | 0.719 | 0.122 | 0.5 | 0.149 |

5d: antibiotic susceptibility of urine samples.

# sample

| Antibiotics | 1 | 2 | 3 | 4 | 5 | 6 |
|---|---|---|---|---|---|---|
| Gentamicin | S | S | S | S | S | S |
| Ampicillin | R | R | S | S | R | S |
| Penicillin G | R | R | S | S | R | S |
| Cefuroxime | S | S | S | S | S | S |
| Orbenil | R | R | R | S | R | R |
| Cephamezine | S | S | S | S | S | S |
| Chloramphenicol | S | S | S | S | S | S |
| Kanamicin | S | S | S | S | S | S |

R = resistant
S = susceptible

### Example 6

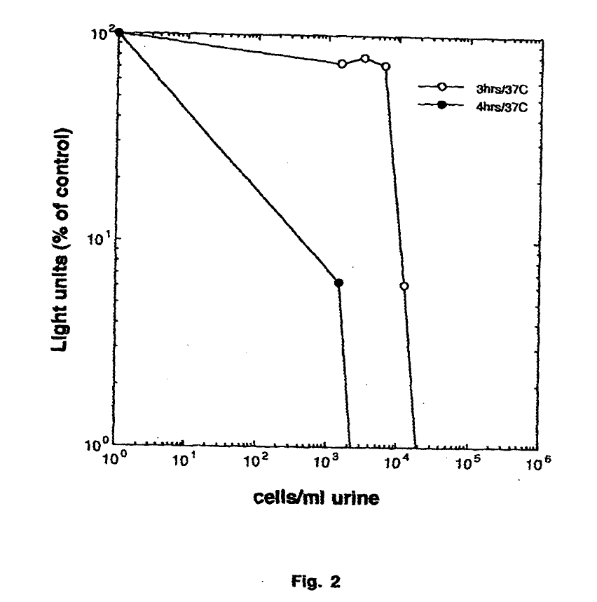

The sensitivity of detection of the assay depends both on the duration of the first incubation, and on the concentration of nutrients (in order to detect a low bacterial concentration a low concentration of nutrients is preferably used). As can be seen in FIG. 2, using a given nutrient concentration, a 3 hr incubation allowed the detection of infecting bacteria at a concentration as low as 10⁴ cells/mL urine, while 4 hrs incubation resulted in the detection of less than 10³ cells/mL urine.

Reference is herein made to a further embodiment of the invention:

By using various dark mutants of luminescent bacteria contaminating microbes in liquids such as water are detected in the following way: the contaminated sample is supplemented with a substrate of an enzyme present on the surface of the contaminating microbe. The end product of that enzymatic reaction serves as a substrate for the mutant luminescent sensor bacteria. A linear correlation was found between the concentration of contaminating microbes in the sample and the light generated by the dark mutants, thus the number of contaminating microbes could be determined.

Three examples of the use of certain mutant luminescent sensor bacteria (dark mutants) for detecting bacteria are presented below.

1. Lipase

Bacteria produce different classes of lipolytic enzyme, including carboxylesterases (EC 3.1.1.1), which hydrolyse small ester-containing molecules at least partly soluble in water, true lipases (EC 3.1.1.3), which display maximal activity towards water-insoluble long-chain triglycerides, and various types of phospho-lipase.

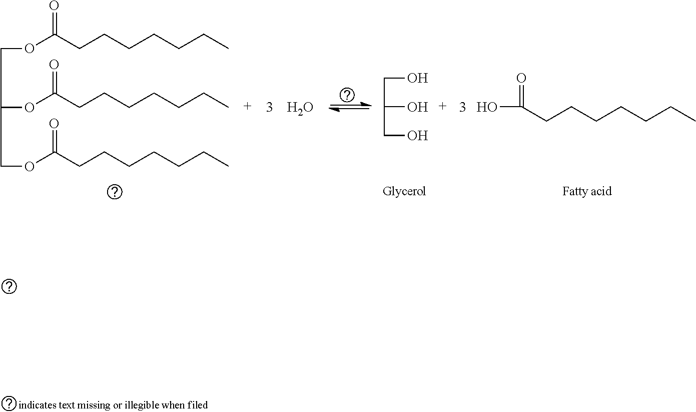

Ulitzur (1978,1979) has shown that the activity of lipase, and phospholipase (A and C) could be determined using a mutant of luminescent bacteria that lacks the ability to generate myristic acid. The luminescence response was shown to be proportional to the amount of added myristic acid over a 100-fold range, down to 10 nM.

It is an embodiment of the invention to disclose a method and means of detecting contaminating cells by measuring the activity of their associated lipases.

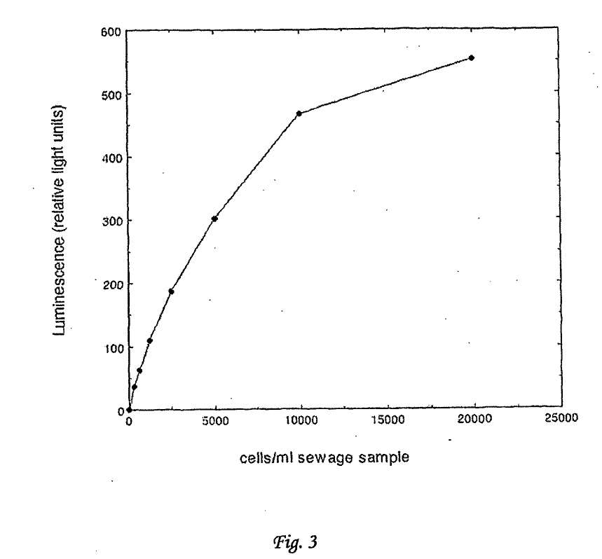

Trimyristin was used as a substrate for lipase and was added to the tested water sample that was spiked with different concentrations of sewage-borne bacteria. After 40 min incubation at 40 degrees Celsius in Tris Buffer pH 8/0.5% NaCl, the mutant M17 of V. harvei cells (final concentration 10⁶/mL) were added together with 100 mg/L glucose, 10 mg/L potassium cyanide, and 2% NaCl with 20 mM phosphate buffer at pH-6. The hydrolyzed myristic acid was determined after 5 min at 28 degrees Celsius by the response of the luminous bacteria either on a continuous basis in the same reaction mixture or alternatively, when the hydrolytic stage is done separately followed by the independent detecting system. Using these procedures it is possible to assay cell-associated lipase activity in contaminated samples. FIG. 3 depicts the linear response between contaminating cells concentration and generated luminescence due to the breakdown of trimyristin to myristic acid that was utilized by the dim mutant. Less than 500 cells could be easily detected using this procedure.

### REFERENCES

1. Ulitzur S. Biochim Biophys Acta. 1979 Feb. 26; 572(2):211-7. A sensitive bioassay for lipase using bacterial bioluminescence.

2. Jean Louis Arpigny and Karl-Erich Jaeger. Biochem. J. (1999) 343, 177-183. Bacterial lipolytic enzymes: classification and properties.

3. Frank Rosenau and Karl-Erich Jaeger. Biochimie Volume 82, Issue 11, November 2000, Pages 1023-1032. Bacterial lipases from Pseudomonas: Regulation of gene expression and mechanisms of secretion.

2. Phospholipase

Outer-membrane PLA (OMPLA; EC 3.1.1.32) is one of the few enzymes present in the outer membrane of Gram-negative bacteria. OMPLA was found E. coli, Helicobacter. pylori, Campylobacter jejuni, Yersinia pestis, Neisseria meningitidis, Neisseria gonorrhoeae, Salmonella. typhimurium, Klebsiella pneumoniae and Proteus vulgaris.

Although OMPLA of Gram-negative bacteria is an integral membrane protein that is embedded in its own substrate in the cell envelope, no enzymatic activity can be detected in normally growing cells. Enzymatic activity can only be induced after severe perturbation of the cell envelope integrity, which occurs during various processes such as phage-induced lysis, temperature shock and colicin secretion. Membrane-perturbing peptides, such as polymyxin B, melittin or cardiotoxin, can also activate the enzyme (Dekker, 2000; Snijder & Dijkstra, 2000).

The advantage of detecting phospholipase activity lies in the fact that it is membrane bound, unlike lipases which usually are secreted to the extra-cellular space.

In this case the substrate used is phosphatidylcholine dimyristate (PCDM) which is hydrolyzed by the OMPLA to trimyristate, which in turn is metabolized by lipase into myristic acid.

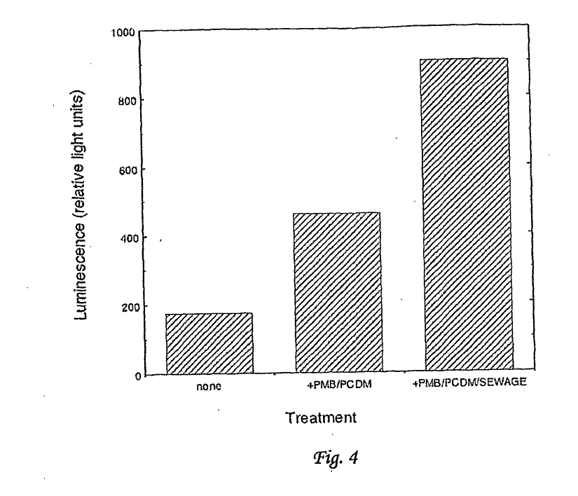

The example shown in FIG. 4 describes a test in which a water sample was preincubated with polymyxin B (PMB-1.25 mg/L) and PCDM (25 mg/L) with and without 10⁵ cells/ml isolated from sewage (a heterogenous population of bacteria). After 45 min at 40 degrees Celsius, the dim mutant bacteria were added together with 2% NaCl/10 mM KH₂PO₄ pH-6. Luminescence was recorded after 2 min at 28 degrees Celsius.

As can be seen in FIG. 2, the presence of the contaminating bacteria resulted in an increase in luminescence due to the generation of myristic acid by the dual enzyme system (phospholipase and lipase).

### REFERENCES

1. Ulitzur S, Heller M. Anal Biochem. 1978 December; 91(2):421-31. A new, fast, and very sensitive bioluminescence assay for phospholipases A and C.

2. Taghrid S. Istivan and Peter J. Coloe. Phospholipase A in Gram-negative bacteria and its role in pathogenesis. Microbiology 152 (2006), 1263-1274

3. Monoamine Oxidase

The principle presented in this invention is not restricted to lipolytic enzymes or to a myristic acid deficient mutant. A different example is described below.

The in vivo luminescence of an aldehyde-requiring mutant of the luminous bacteria Vibrio harveyi (M42) was shown to increase' dramatically upon the addition of long-chain aliphatic aldehydes (C8-C16). The intensity of this luminescence is linearly related to aldehyde concentration. This property could be utilized for the determination of monoamine oxidase activity using n-decylamine as substrate, which is converted by the enzyme monoamine oxidase to n-decylaldehyde (Ulitzur 1985).

Monoamine oxidase was found in some strains of the family Enterobacteriaceae, such as Klebsiella, Enterobacter, Escherichia, Salmonella, Serratia, and Proteus. It is another core object of this invention to disclose means and methods of detecting the presence of the aforementioned bacteria in liquids such as water by measuring the activity of membrane bound monamine oxidase.

### REFERENCES

1. Tenne M, Finberg J P, Youdim M B, Ulitzur S. J Neurochem. 1985 May; 44(5):1378-84. A new rapid and sensitive bioluminescence assay for monoamine oxidase activity.

2. Y Murooka, N Doi, and T Harada. Appl Environ Microbiol. 1979 38 (4): 565-569. Distribution of membrane-bound monoamine oxidase in bacteria.

Detection of Bacteria in Water

It is a core principle of the present invention to provide means and methods of detecting bacteria in water. The following example conveys essentials of such means and methods. It is acknowledged that sufficient disclosure is herein presented so as to enable a person skilled in the art to envisage and carry out variations of the example presented without undue experimentation.

### EXAMPLE

Heterotrophic bacteria isolated from mineral water and soil were inoculated into boiled tap water and serially diluted with Assay buffer (containing salts and basic minerals). All the tubes were then spiked with 10 microgram/L mix of yeast extract and glucose and incubated for one hour at 28 degrees Celsius. Dim and starved luminescent bacteria (Vibrio harveyi) cells suspended in salt buffer (inositol-5%; MgSO₄-50 mM; NaCl-1.7%; MOPS-20 mM_) were dispensed into each tube at a final cell concentration of 10⁶/mL. Following an additional incubation of about 2 hours at 28 degrees Celsius, luminescence was recorded. As can be seen in FIG. 3 the higher the concentration of heterotrophic bacteria in the sample the lower the light level detected. Using this approach, one could detect about 100 cells in 1 Ml sample.

Automation

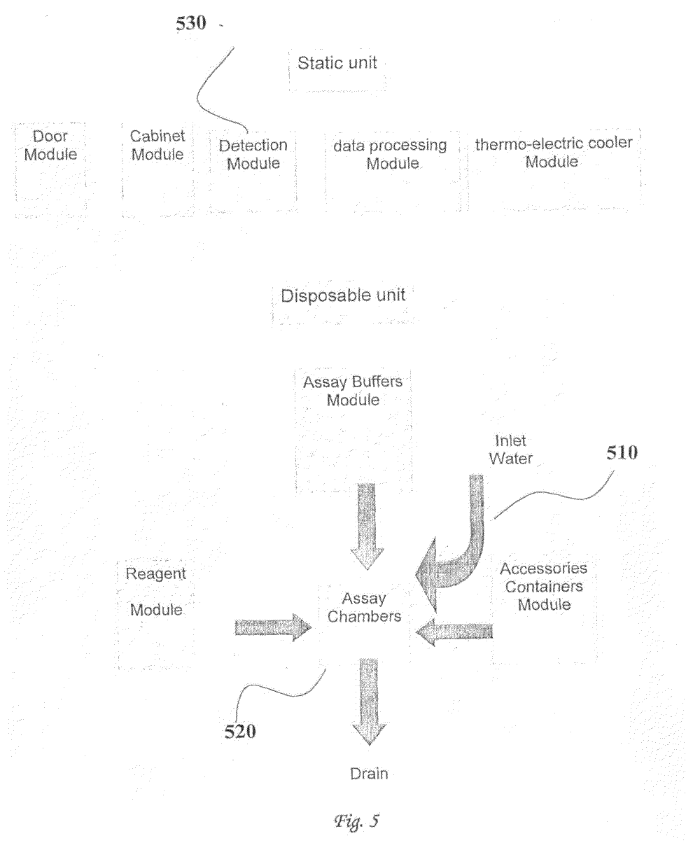

A preferred embodiment of the present invention, providing automatic means and methods for detecting microbes in a fluid is herein described in FIG. 5:

Inlet water 510 is sampled into the temperature-controlled assay chamber 520 (volume-1 mL) into which the concentrated assay buffer is injected together with the defined concentration of the chosen nutrient (e.g., glucose). The solution is incubated for a given time at the optimal temperature to allow the bacteria in the sample to digest the provided metabolites. At the next stage, the sensor (tester) bacteria which are kept in a solution in a temperature-controlled chamber are injected into the assay chamber and further incubated for 1-2 hrs. The assay chamber is then exposed to the photomultiplier detection 530 module in order to record the emitted luminescence. The data is stored in the microprocessor within the instrument and is compared to data obtained with the control set. The control set is run in parallel to the sample set since the instrument holds a dual assay chamber system. The control cycle is basically the same except for the water source which is sterile reference water. The alarm control in the device is designed to send out a warning signal whenever the light level in the sample set is lower than that obtained with the control set.

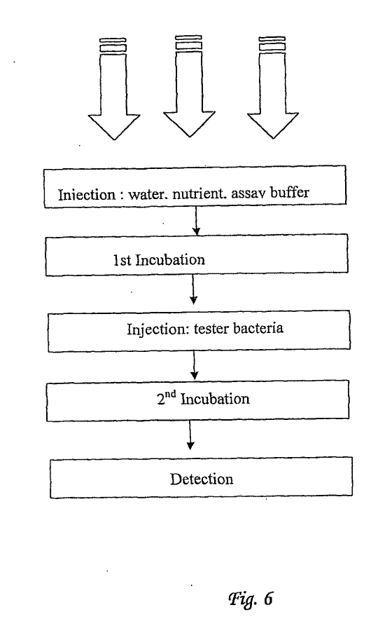

Reference is now made to the block diagram of FIG. 6 schematically illustrating stages in the aforementioned method, as described above.

It is well within the scope of the present invention to provide and disclose automatic means and methods for determining the presence of infecting microbes in a liquid, especially bacteria.

It is also well within the scope of the present invention to provide and disclose automatic means and methods for determining the susceptibility of microbes in a liquid specimen.

It is also well within the scope of the present invention to provide and disclose automatic means and methods for determining a minimum inhibitory concentration of a substance and of microbes, especially bacteria in a liquid.

Moreover it is well within the scope of the present invention to provide and disclose automatic means and methods for determining whether bacteria in a liquid specimen are Gram positive or Gram negative.

1-37. (canceled)

38. A method for determining the presence of infecting microbes in a liquid, said method comprising steps of:
a. obtaining a microbial analyzer;
b. introducing said liquid to the sample container of said microbial analyzer, said sample container further containing nutrients;
c. carrying out a first incubation of said specimen;
d. adding bioluminescent tester microbes to said specimen;
e. carrying out a second incubation of said specimen; and
f. measuring bioluminescence signal thereby determining said presence and/or concentration of said infecting microbes.

39. The method of claim 38, wherein the intensity of said measured signal is positively correlated with the concentration of nutrients in the inoculum at the beginning of said second incubation.

40. The method of claim 38, wherein said infecting microbe is a bacterium.

41. The method of claim 38, wherein said tester microbe is a bacterium.

42. The method of claim 38, further comprising steps of selecting said liquid from a group consisting of mains supply water, drinking water, tap water, bottled water, a beverage, food process water, medicinal process water, reclaimed water, agricultural water and irrigation water.

43. The method of claim 42, wherein said method comprises steps of concentrating said microbes.

44. The method of claim 42, wherein said method comprises steps of preconcentrating said microbes.

45. The method according to claim 41, wherein said tester microbe is a bacteria of the GlnA mutant strain of E. coli (ET 12558) carrying the Lux-I deleted lux system of Vibrio fischeri.

46. The method according to claim 38, wherein said method comprises steps of selecting said nutrients from a group consisting of amino acids, peptides, vitamins, fatty acids, nucleotides and sugars further wherein said tester bacteria are additionally characterised as luminescent mutants lacking the ability to generate said selected nutrient.

47. The method according to claim 38, wherein said method further comprises steps of:
a. adding to said specimen a reagent comprising tester bacteria of a luminescent mutant, said mutant further characterized by only luminescing in the presence of a breakdown product of a substrate of a surface enzyme of said infecting microbes, said luminescing in proportion to concentration of said breakdown product at the beginning of said second incubation; and
b. adding to said specimen said substrate of said infecting microbes.

48. The method according to claim 47, wherein said method comprises steps of adding to said specimen a reagent comprising aldehyde-requiring or myristic acid-requiring mutants of Vibrio harveyi; said mutant characterised by luminescing in the presence of a breakdown product of a substrate of a surface enzyme of said infecting microbes, said luminescing in proportion to concentration of said breakdown product at the beginning of said second incubation, and adding to said specimen said substrate of said infecting microbes wherein said breakdown product is defined as a substrate of a surface enzyme selected from a group consisting of lipase, phospholipase A, phospholipase C and monoamine oxidase or any enzyme pre-released from the infecting cells by osmotic shock.

49. A method for determining the susceptibility of microbes in a liquid specimen to a substance and determining a minimal concentration of the substance to which the bacteria are susceptible to a substance, said method comprising steps of obtaining a kit comprising
a. a microbial analyzer, said analyzer further comprising
i. a reaction chamber for carrying out a first incubation and a second incubation of said liquid specimen
ii. reagent containers for containing and introducing reagents, buffers and nutrients into said reaction chamber
iii. a light detector adapted for detecting luminescence during said incubations;
b. a reagent, wherein said reagent comprises tester bacteria of an E. coli nutrient-requiring mutant strain carrying the Lux-I deleted lux system of Vibrio fischeri, said strain characterized by luminescing in proportion to the concentration of said nutrients remaining at the beginning of said second incubation.
c. adding said substance to the specimen.
d. determining the concentration of microbes in said specimen by the method defined in claim 38 and
e. comparing the concentration of the microbes in the specimen to the concentration in a control specimen to which the substance was not added, a lower concentration of microbes in the specimen than that in the control specimen indicating susceptibility of said microbes to said substance and then determining a minimal concentration of the substance to which the bacteria are susceptible.

50. The method according to claim 49, said method further comprising steps of adding a said substance that selectively inhibits Gram positive or Gram negative bacteria to said first or second incubation wherein said method further comprises steps of adding cetyltrimethyl-ammonium bromide to said specimen and determining said microbes susceptibility to said cetyltrimethyl-ammonium bromide.

51. The method according to claim 49, wherein said nutrients are selected from a group consisting of amino acids, peptides, vitamins, nucleotides and sugars further wherein said tester bacteria of an naturally selected luminescent bacteria mutant or E. coli strain carrying the Lux-I deleted lux system of Vibrio fischeri are additionally characterised as mutants lacking the ability to generate said selected nutrient.

52. The method according to claim 49, wherein said kit further comprises:
a. a reagent comprising tester bacteria of a luminescent dark mutant said mutant further characterized by only luminescing in the presence of a breakdown product of a substrate of a surface enzyme of said infecting microbes, said luminescing in proportion to concentration of said breakdown product at the beginning of said second incubation; and
b. said substrate of said infecting microbes, and
wherein said breakdown product is defined as a substrate of an enzyme pre-released from the infecting cells by osmotic shock further wherein said mutant is a myristic acid-requiring mutant of Vibrio harveyi or an aldehyde-requiring mutant of Vibrio harveyi.

53. A system for determining the presence of an infecting microbe in a liquid specimen, said system comprising:
a. a microbial analyzer, said analyzer further comprising
i. a reaction chamber for carrying out a first incubation and a second incubation of said liquid specimen
ii. reagent containers for containing and introducing reagents, buffers and nutrients into said reaction chamber
iii. a light detector adapted for detecting bioluminescence during said incubations; and
b. a reagent, wherein said reagent comprises tester bacteria of an E. coli nutrient-requiring mutant strain carrying the Lux-I deleted lux system of Vibrio fischeri, said strain characterized by luminescing in proportion to the concentration of said nutrients remaining at the beginning of said second incubation.

54. The system according to claim 53, wherein said nutrients are selected from a group consisting of amino acids, peptides, fatty acids, vitamins, nucleotides and sugars further wherein said sensor luminescent bacteria are additionally characterized as mutants lacking the ability to generate said selected nutrient.

55. The system according to claim 53, wherein said system further comprises:
a. a reagent comprising tester bacteria of a luminescent dark mutant said mutant further characterized by only luminescing in the presence of a breakdown product of a substrate of a surface enzyme of said infecting microbes, said luminescing in proportion to concentration of said breakdown product at the beginning of said second incubation; and
b. said substrate of said infecting microbes and said luminescent dark mutant is a myristic acid-requiring mutant of Vibrio harveyi or an aldehyde-requiring mutant of Vibrio harveyi, further wherein said surface enzyme is selected from a group consisting of lipase, phospholipase A, phospholipase C and monoamine oxidase.

56. The system according to claim 53, wherein said system is provided with any means of automatic or semi automatic means of detecting microbes in water selected from the group consisting of means for sample preparation, reagent preparation, first and/or second incubations and light detection and further wherein said system is provided with means of data processing.

57. The system according to claim 53, wherein said reagent further comprises:
a. tester bacteria of a luminescent mutant, said mutant further characterized by only luminescing in the presence of a breakdown product of a substrate of a surface enzyme of said infecting microbes, said luminescing in proportion to concentration of said breakdown product at the beginning of said second incubation; and
b. said substrate of said infecting microbes.
